(19) United States
(12) Patent Application Publication
(10) Pub. No.: US 2023/0058262 A1
(43) Pub. Date: Feb. 23, 2023

(54) ULTRA LONG-LASTING MAKE-UP FOUNDATION

(71) Applicant: L V M H RECHERCHE, Saint -Jean De Brave (FR)

(72) Inventors: Anne-Sophie Rattina, Saint Jean De Braye (FR); Laurent Neau, Saint Jean De Braye (FR); Marine Martinez, Saint Jean De Braye (FR); Valérie Alard, Saint Jean De Braye (FR)

(21) Appl. No.: 17/787,237

(22) PCT Filed: Dec. 18, 2020
(86) PCT No.: PCT/FR2020/052539

§ 371 Date: Jun. 17, 2022

(30) Foreign Application Priority Data
Dec. 19, 2019 (FR) 1914850

(51) Int. Cl. A61K 8/06; A61Q 1/02; A61K 8/87; A61K 8/31; A61K 8/891; A61K 8/29; A61K 8/19; A61K 8/58

(52) U.S. Cl. CPC A61K 8/064; A61Q 1/02; A61K 8/87; A61K 8/31; A61K 8/891; A61K 8/29; A61K 8/19; A61K 8/585; A61K 2800/43; A61K 2800/594

(57) ABSTRACT

The present invention relates to a cosmetic make-up composition for the skin in the form of a water-in-oil (W/O) emulsion comprising in a physiological acceptable medium at least:
a) an aqueous polyurethane dispersion,
b) a hydrophobic film-forming polymer,
c) one or more volatile hydrocarbon oils, preferably in an amount of at least 10 wt % relative to the total weight of the composition,
d) one or more absorbent fillers having an oil uptake greater than or equal to 30 ml/100 g and
e) pigments.

### CROSS REFERENCE TO RELATED APPLICATIONS

This application is a U.S. National Stage of International Patent Application No. PCT/FR2020/052539 filed Dec. 18, 2020, which claims the benefit of priority of French Patent Application No. 1914850 filed Dec. 19, 2019, the respective disclosures of which are each incorporated herein by reference in their entireties.

### FIELD OF THE INVENTION

The present invention relates to a make-up composition for keratinous materials, in particular the skin, having improved wearing of the make-up under extreme conditions (sebum, sweat). It is referred to as ‘ultra-long lasting’ make-up.

### PRIOR ART

Cosmetic make-up compositions, such as foundations for example, are commonly used to bring colour and an aesthetic effect to the skin, in particular to the face. These compositions generally comprise oils (comfort), pigments (colour) and fillers (mattness). However, one of the problems with make-up products is the wear of the make-up over time, in particular good wear of colour, mattness, and/or homogeneity. This problem is even more important for skin having increased sebum secretion (oily skin and/or skin subject to intense physical exercise) and skin subject to extreme conditions of temperature and/or humidity.

It is known from the prior art to use polymers providing wear over the course of the day. However, foundations on the market that are positioned as long-lasting are often uncomfortable or deceptive in terms of wear under extreme conditions (heat and humidity). There is still a need therefore to provide cosmetic make-up compositions for the skin having an improved mattness and wear of mattness and/or colour, in particular resistant to extreme conditions such as hot and/or humid atmospheres and sweating conditions associated with intense physical exercise (e.g. sport).

The applicant has observed that the combined use of two stability polymers, one in the aqueous phase for wear and comfort and the other in the oily phase for the resistance to perspiration and to sebum, with a large amount of volatile oils, preferably hydrocarbons, and a selection of absorbent fillers for their oil absorption capacity, can respond to this need.

### DISCLOSURE OF THE INVENTION

An aim of the invention is therefore to propose a cosmetic make-up composition for the skin, in particular a foundation in the form of a water-in-oil emulsion, having an improved wear and homogeneity, intended in particular for extreme conditions (hot and humid climates or intense physical exercise).

“Wear” corresponds to make-up that is still present and satisfactory at the end of the day. Good wear corresponds to a foundation which does not require touching up over the course of the day.

“Uniformity or homogeneity” is the ability of the foundation to remain uniform throughout the day, in other words it does not amalgamate in patches, does not form areas of different thicknesses and is not absorbed in some places at the end of the day.

To this effect, according to a first aspect of the invention, a cosmetic make-up composition for the skin is proposed in the form of a water-in-oil (W/O) emulsion comprising, in a physiologically acceptable medium, at least:

a) an aqueous polyurethane dispersion,
b) a hydrophobic film-forming polymer,
c) one or more volatile hydrocarbon oils, preferably in an amount of at least 10 wt % relative to the total weight of the composition,
d) one or more absorbent fillers having an oil uptake greater than or equal to 30 ml/100 g and
e) pigments.

According to a particular embodiment, the composition of the invention comprises an amount of non-volatile oil less than or equal to 5 wt %, in particular less than or equal to 2 wt %, or even less than or equal to 1 wt % relative to the total weight of said composition, or even is devoid of non-volatile oils.

The invention also relates to a cosmetic method for make-up of the skin comprising the application on the skin of a composition as defined in the invention.

According to a particular embodiment, according to the method of the invention, the composition is applied to oily skin and/or skin subject to hot and/or humid atmospheric conditions and/or sweating conditions associated with intense physical exercise.

In the context of the invention, “oily skin” shall mean skin having a shiny complexion and/or a perception of discomfort, surface irregularities, in particular dilated follicle orifices or pores, imperfect skin texture and/or skin having poor make-up stability.

“Intense physical exercise” shall mean physical exercise stimulating perspiration and the secretion of sebum, for example the practice of a sport.

Other features, aims and advantages of the invention will emerge from the following description, which is given purely by way of illustration and not being limiting.

### DETAILED DESCRIPTION OF THE INVENTION

A first object of the invention is a cosmetic make-up composition for the skin in the form of a water-in-oil (W/O) emulsion comprising, in a physiologically acceptable medium, at least:

a) an aqueous polyurethane dispersion,
b) a hydrophobic film-forming polymer,
c) one or more volatile hydrocarbon oils, preferably in an amount of at least 10 wt % relative to the total weight of the composition,
d) one or more absorbent fillers having an oil uptake greater than or equal to 30 ml/100 g and
e) pigments.

In the context of the invention, “physiologically acceptable medium” shall mean that the medium is compatible with human keratinous materials and has a pleasant appearance (touch, colour and/or smell) without generating discomfort on application to said keratinous materials.

In the context of the invention, “keratinous materials” shall mean the skin of the face and/or of the neck.

### Aqueous Phase

The aqueous phase of the composition according to the invention generally represents 1 to 70 wt %, in particular 30 to 60 wt %, relative to the total weight of said composition.

The aqueous phase comprises water and optionally a water-soluble solvent.

In the context of the invention, “water-soluble solvent” means a compound that is liquid at room temperature and miscible with water (miscibility in water greater than 50 wt % at 25° C. and atmospheric pressure). These include, in particular:
monoalcohols that are less than C₁-C₅ such as ethanol, isopropanol and the mixtures thereof, preferably ethanol;
C₂-C₈ glycols such as ethylene glycol, propylene glycol, 1,3-butylene glycol, dipropylene glycol, and the mixtures thereof;
C₂-C₃₂ polyols such as glycerol, polyglycerols, polyethylene glycols, and the mixtures thereof, and the mixtures thereof.

It can also comprise hydrophilic gelling agents, antioxidants, preservatives and the mixtures thereof.

Hence, a cosmetic composition according to the invention further comprises at least one water-soluble solvent chosen among the lower C₁-C₅ monoalcohols, C₂-C₈ glycols, C₂-C₃₂ polyols and the mixtures thereof, preferably in a total amount ranging from 5 to 25 wt %, in particular 8 to 20 wt % relative to the total weight of said composition.

According to a particular and preferred embodiment, the composition of the invention will comprise at least ethanol, preferably in an amount ranging from 5 to 15 wt % relative to the total weight of the composition, conferring a fresh effect.

The composition will advantageously further comprise at least polyols and/or glycols in a total amount ranging from 3 to 12 wt %, in particular 5 to 10 wt % relative to the total weight of the composition, for a moisturising effect promoting the obtaining of a radiant complexion (‘glowy’ effect) without oily effect.

### Aqueous Dispersion of Polyurethane

An aqueous dispersion of polyurethane according to the invention is as described, in particular, in patent application EP1970391 filed in the name of Bayer MaterialScience LLC.

in particular, the dispersed polyurethane comprises the reaction products of:

A) a prepolymer according to formula

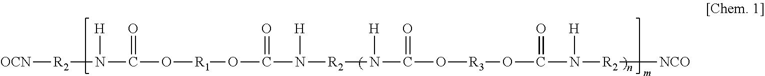

wherein
R₁ represents a bivalent radical of a dihydroxyl compound, in particular a hydrocarbon radical derived from a polyester polyol, and in particular from a polyester diol,
R₂ represents a hydrocarbon radical derived from an aliphatic or cycloaliphatic polyisocyanate,
R₃ represents in a hydrocarbon radical derived from a low molecular weight diol, optionally substituted by ionic groups,
n has a value of 0 to 5, and
m is >1;
B) at least one chain extender according to the formula:

H₂N—R₄—NH₂ [Chem. 2]

wherein
R₄ represents an alkylene or alkylene oxide radical that is not substituted by ionic or potentially ionic groups; and
C) at least one chain extender according to the formula:

—H₂N—R₅—NH₂ [Chem. 3]

wherein
R₅ represents an alkylene radical substituted by ionic or potentially ionic groups.

According to a particular embodiment:
the radical R1 is obtained by polycondensation of at least one dicarboxylic acid with at least one diol, said dicarboxylic acid preferably being chosen among adipic acid, the diol preferably being chosen among hexanediol, neopentyl glycol, and the mixtures thereof;
the radical R2 is derived from an aliphatic or cycloaliphatic polyisocyanate, and in particular a diisocyanate chosen among 1,6-hexamethylene diisocyanate, isophorone diisocyanate and dicyclohexylmethane diisocyanate, and the mixtures thereof;
the radical R3 is derived from neopentyl glycol;
the chain extender according to formula Chem. 2 is chosen among ethylene diamine, diethanolamine and the mixtures thereof; and
the chain extender according to formula Chem. 3 is chosen among the diaminosulfonates, and is preferably the sodium salt of N-(2-aminoethyl)-2-aminoethanesulfonic acid.

### Prepolymer A)

### Radical R1

Suitable compounds for providing the polyhydroxyl, preferably dihydroxyl, radical R1 are divalent compounds, preferably with two hydroxy groups and having average molecular weights of approximately 700 to approximately 16,000, and preferably approximately 750 to approximately 5000.

These compounds are preferably chosen among the polyester polyols, preferably the polyester diols, and the mixtures thereof.

The one or more polyester diols can generally be prepared from dicarboxylic acids or aliphatic, cycloaliphatic or aromatic polycarboxylic acids or the anhydrides thereof (e.g. succinic, fumaric, glutaric, 2,2-dimethylglutaric, adipic, itaconic, pimelic, suberic, azelaic, sebacic, maleic, malonic, 2,2-dimethylmalonic, nonanedicarboxylic, decanedicarboxylic, dodecanedioic, 1,3-cyclohexanedicarboxylic, 1,4-cyclohexanedicarboxylic, 2,5-norborane dicarboxylic, diglycolic, thiodipropionic, 2,5-naphthalenedicarboxylic, 2,6-naphthalene-dicarboxylic, phthalic, terephthalic, isophthalic, oxanic, o-phthalic, tetrahydrophthalic, hexahydrophthalic or trimellitic acid; and the anhydride of o-phthalic, trimellitic or succinic acid or a mixture of these), and dihydric alcohols, such as diols chosen among the aliphatic, alicyclic and aromatic diols (e.g. ethane diol, ethylene glycol, diethylene glycol, triethylene glycol, trimethylene glycol, tetraethylene glycol, 1,2-propanediol, dipropylene glycol, tripropylene glycol, tetrapropylene glycol, 1,3-propanediol, 1,4-butanediol, 1,3-butanediol, 2,3-butanediol, 1,5-pentanediol, 1,6-hexanediol, 2,2-dimethyl-1,3-propanediol, 1,4-dihydroxycyclohexane, 1,4-dimethylolcyclohexane, cyclohexanedimethanol, 1,8-octanediol, 1,10-decanediol, 1,12-dodecanediol, neopentyl glycol or the mixtures thereof).

The dicarboxylic acid is preferably adipic acid.

The polyester diols can also be chosen among the homopolymers or copolymers of lactones, which are preferably obtained by addition reactions of lactones or mixtures of lactones, such as butyrolactone, c-caprolactone and/or methyl-c-caprolactone with polyfunctional initiating molecules, preferably suitable difunctionals, such as the dihydric alcohols cited above, for example. The corresponding polymers of c-caprolactone are preferred.

The polyester polyol radical R₁, preferably polyester diol, can be advantageously obtained by polycondensation of dicarboxylic acids, such as adipic acid, with polyols, in particular diols, such as hexanediol, neopentyl glycol, and the mixtures thereof.

### Radical R2

Appropriate polyisocyanates for providing the hydrocarbon radical R₂ comprise organic diisocyanates having a molecular weight of approximately 112 to 1000, and preferably approximately 140 to 400.

Preferred diisocyanates are those represented by the general formula R₂(NCO)₂ indicated above, wherein R₂ represents a divalent aliphatic hydrocarbon group comprising 4 to 18 carbon atoms, a divalent cycloaliphatic hydrocarbon group comprising 5 to 15 carbon atoms, a divalent araliphatic hydrocarbon group comprising 7 to 15 carbon atoms or a divalent aromatic hydrocarbon group comprising 6 to 15 carbon atoms. Examples of suitable organic diisocyanates include tetramethylene diisocyanate, 1,6-hexamethylene diisocyanate, dodecamethylene diisocyanate, cyclohexane-1,3-diisocyanate and cyclohexane-1,4-diisocyanate, 1-isocyanato-3-isocyanatomethyl-3,5,5-trimethylcyclohexane (isophorone diisocyanate or IPDI), bis-(4-isocyanatocyclohexyl)-methane, 1,3-bis(isocyanatomethyl)-cyclohexane and 1,4-bis(isocyanatomethyl)-cyclohexane, bis-(4-isocyanato-3-methyl-cyclohexyl)-methane. Mixtures of diisocyanates can of course be used. Preferred diisocyanates are aliphatic and cycloaliphatic diisocyanates. Particularly preferred is 1,6-hexamethylene diisocyanate, isophorone diisocyanate and dicyclohexylmethane diisocyanate and the mixtures thereof.

### Radical R3

The use of low molecular weight diols, R₃ can enable a stiffening of the polymer chain and is optional. The expression “low molecular weight diols” signifies diols having a molecular weight of approximately 62 to 700, preferably 62 to 200. They can contain aliphatic, alicyclic or aromatic groups. The preferred compounds only contain aliphatic groups. The diols used preferably have up to 20 carbon atoms and can be chosen among ethylene glycol, diethylene glycol, propane-1,2-diol, propane-1,3-diol, butane-1,4-diol, butylene-1,3-glycol, neopentyl glycol, butyl ethyl propanediol, cyclohexanediol, le 1,4-cyclohexanedimethanol, hexane-1,6-diol, bisphenol A (2,2- bis(4-hydroxyphenyl)propane), bisphenol A hydrogen (2,2-bis(4-hydroxycyclohexyl)propane) and the mixtures thereof. Preferably, R₃ is derived from neopentyl glycol.

Optionally, the low molecular weight diols can contain ionic or potentially ionic groups. Appropriate low molecular weight diols containing ionic or potentially ionic groups are those disclosed in U.S. Pat. No. 3,412,054. Preferred compounds comprise dimethylol butanoic acid (DMBA), dimethylol propionic acid (DMBA) and caprolactone-polyester diol containing carboxyl. Low molecular weight diols containing ionic or potentially ionic groups are preferably not used.

### Chain Extenders B) and C)

The prepolymer chain A) is extended using two classes of chain extender B) and C) described below.

### Chain Extenders (First Class) B)

Compounds of the first class of chain extension have the formula:

H₂N—R₄—NH₂ [Chem. 4]

wherein R₄ represents an alkylene or alkylene oxide radical that is not substituted by ionic or potentially ionic groups.

Thus, the chain extender can be chosen among:
alkylenediamines such as hydrazine, ethylenediamine, propylenediamine, 1,4-butylenediamine and piperazine.
alkylene oxide diamines, such as dipropylamine-diethyleneglycol (DPA-DEG), 2-methyl-1,5-pentanediamine, hexanediamine, isophoronediamine and 4,4-methylenedi(cyclohexylamine), and the series of DPA-etheramines, comprising dipropylamine propyleneglycol, dipropylamine dipropylene glycol, dipropylamine tripropylene glycol, dipropylamine polypropylene glycol, dipropylaminethylene glycol, dipropylamine polyethylene glycol, dipropylamine-1,3-propanediol, dipropylamine-2-methy1-1,3-propanediol, dipropylamine-1,4-butanediol, dipropylamine-1,3-butanediol, dipropylamine-1,6-hexanediol and dipropylamine cyclohexane-1,4-dimethanol, and the mixtures thereof.

Preferably, the chain extender of the first class is chosen among ethylenediamine, diethanolamine and the mixtures thereof.

### Chain Extenders (Second Class) C)

The second class of chain extenders comprises compounds having the formula:

H₂N—R₅—NH₂ [Chem. 5]

wherein R₅ represents an alkylene radical substituted by ionic or potentially ionic groups. These compounds have an ionic or potentially ionic group and two groups which are reactive with isocyanate groups.

The ionic group or the potentially ionic group can be chosen from the group consisting of tertiary or quaternary ammonium groups, groups that can be converted into such a group, a carboxyl group, a carboxylate group, a sulfonic acid group and a sulfonate group. Specific compounds comprise diaminosulfonates, such as, for example, the sodium salt of N-(2-aminoethyl)-2-aminoethanesulfonic acid (AAS) or the sodium salt of N-(2-aminoethyl)-2-aminopropionic acid.

Preferably, R₅ represents an alkylene radicals substituted by sulfonic acid or sulfonate groups, more preferably the sodium salt of N-(2-aminoethyl)-2-aminoethanesulfonic acid (AAS).

### Chain Terminations

The polyurethane according to the invention can also comprise compounds which are located in each case at the ends of chains and terminate said chains. These chain terminations can derive from compounds having the formula

### [Chem. 6]

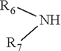

wherein R6 represents a hydrogen atom or an alkylene radical optionally having a hydroxyl end and R7 represents an alkylene radical optionally having a hydroxyl end. Suitable compounds comprise compounds such as monoamines, in particular secondary monoamines or monoalcohols. Examples include: methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, diethanolamine and suitable substituted derivatives thereof, amide-amines of primary diamines and monocarboxylic acids, monocetimes of primary diamines, primary/tertiary amines such as N,N-dimethylamino-propylamine and the like. The chain termination alcohols can be chosen among the C₁-C₁₀ alcohols, such as methanol, butanol, hexanol, 2-ethylhexylic alcohol, isodecylic alcohol, and the mixtures thereof. Aminoalcohols such as aminomethyl propanol (AMP) are also suitable.

In an embodiment of the invention, diethyleneglycol is used in order to obtain polyurethane either as a low molecular weight diol or as part of the non-ionic chain extender via the use of dipropylamine-diethyleneglycol. In the case where the diethyleneglycol is used as low molecular weight diol, then, preferably, DPA-DEG is not used as non-ionic chain extender. Similarly, if DPA-DEG is used as non-ionic chain extender, then, preferably, diethyleneglycol is not used as low molecular weight diol.

The aqueous dispersion of polyurethane according to the invention can have an amount of solids (dry matter) of 20 to 60 wt %, preferably 28 to 50 wt %, and in particular 39 to 42 wt %.

Such dispersions of polyurethanes are, for example, polyurethane 34 sold under the tradenames Baycusan® C1000, C1001 by BAYER, and polyurethane 35 sold under the tradename Baycusan® C1004 by BAYER (COVESTRO).

A preferred aqueous dispersion of polyurethane according to the invention is that marketed under the name Baycusan® C1004 by BAYER (COVESTRO), under the name INCI ‘Polyurethane-35’ or ‘Polyurethane-35 and water’. This dispersion comprises 41% polyurethane in dry matter and 59 wt % water.

According to a particular embodiment, the dispersed polyurethane is present in the composition with a dry matter content ranging from 0.2 to 20 wt %, in particular 0.5 to 15 wt %, preferably 1 to 10 wt %, more preferably 1 to 7 wt % of dry matter (polyurethane) relative to the total weight of said composition.

### Oily Phase

The composition according to the invention comprises at least one continuous oily phase.

The term “oily phase” shall mean an oil or a mixture of mutually miscible oils.

In the context of the invention, the term “oil” shall mean a fatty substance that is not soluble in water and that is liquid at 25° C. and atmospheric pressure.

An oily phase according to the invention may comprise hydrocarbon oils, silicone hydrocarbon oils, fluorinated or non-fluorinated oils, and the mixtures thereof.

These oils can be volatile or non-volatile, vegetable, mineral or synthetic.

In the context of the invention, ‘volatile oil’ shall mean an oil with volatile nature defined by the protocol described below and in the illustrative examples.

In the context of the invention, ‘non-volatile oil’ shall mean an oil that does not respond to the volatility criteria defined above.

In the context of the invention, “hydrocarbon oil” shall mean an oil containing mainly hydrogen and carbon atoms.

In the context of the invention, ‘silicone oil’ shall mean an oil comprising at least one silicon atom, and in particular a Si—O group.

In the context of the invention, ‘fluorinated oil’ shall mean an oil comprising at least one fluorine atom.

### Volatile Oils

In the context of the invention, volatile oil shall mean an oil having lost more than 20 wt % of its mass at 15 minutes, more than 40 wt % of its mass at 30 minutes and more than 70 wt % of its mass at 60 minutes, according to the protocol described below.

Protocol for monitoring the volatility of oils:

20 mg of the oil to be studied is weighed on a plate of PMMA of 5 cm×5 cm using a micropipette and a precision balance. This material is spread by finger over the entire plate. Said plate is then deposited in a thermostatic ventilated chamber at 25° C. and 50% humidity. For this material, the test is performed 3 times in order to calculate a standard deviation and to have an idea of the repeatability of the method.

The mass loss during drying is measured after 15 minutes, 30 minutes and 60 minutes. The mass loss is expressed according to the following calculation:

$$\% mass loss = \frac{{m}_{t 0} - {m}_{tx}}{{m}_{t 0}} \times 100 \qquad [ Math . 1 ]$$

with mtx corresponding to the mass remaining at the measurement time (t15 min, t30 min or t60 min) and mt0 corresponding to the initially applied mass.

The volatility measurements of the oils are expressed in time (in minutes).

A person skilled in the art will thus be able to define suitable oils for the invention on the basis of this test for monitoring its mass loss as a function of time.

The composition of the invention comprises one or more volatile oils, in a total amount of at least 10 wt % and ranging in particular from 10 to 40 wt %, preferably 15 to 25 wt % relative to the total weight of said composition.

The large amount of volatile phase, and in particular of volatile oils makes it possible to have a light composition that is easily applied on the skin; the volatile oils participate in the placing of the film on the skin during application and, when they evaporate, they leave the film adhering to the skin, with a sensation of naked skin, without material effect or masking effect on the skin.

The volatile oils can be chosen among silicone oils, hydrocarbon oils and the mixtures thereof.

Volatile silicone oils include, in particular, linear or cyclic volatile silicone oils and the mixtures thereof. This includes, in particular, silicone oils such as dimethicones (polydimethylsiloxanes) the viscosity of which ranges from 0.5 to 6 cSt, alkyl trisiloxanes and cyclomethicones. These include, for example, octamethyl cyclotetrasiloxane, decamethyl cyclopentasiloxane, dodecamethyl cyclohexasiloxane, hexamethyl disiloxane, heptamethyl hexyltrisiloxane, heptamethyloctyl trisiloxane, octamethyl trisiloxane, 1,1,1,3,5,5,5-heptamethyl-3-(trimethylsilyloxy)trisiloxane (alternatively called methyl trimethicone), decamethyl tetrasiloxane, dodecamethyl pentasiloxane, and the mixtures thereof.

Volatile hydrocarbon oils include, in particular, C₉-C₁₃ linear alkanes, C₈-C₁₆ branched alkanes, and the mixtures thereof.

According to a particular embodiment, the volatile hydrocarbon oils are chosen among the volatile linear alkanes, isododecane, and the mixtures thereof.

C₉-C₁₃ Volatile Linear Alkanes

By way of example, linear alkanes suitable for the invention may include n-nonane (C9), n-decane (C10), n-undecane (C11), n-dodecane (C12), n-tridecane (C13), and the mixtures thereof. According to a particular embodiment, the volatile linear alkane is chosen from n-nonane, n-undecane, n-dodecane, n-tridecane, and the mixtures thereof. According to a preferred embodiment these include the mixtures of n-undecane (C11) and n-tridecane (C13) such as those sold under the name CETIOL by BASF, and the mixtures of n-dodecane (C12) and n-tetradecane (C14) such as those sold under the name VEGELIGHT by BIOSYNTHIS, and the mixtures thereof.

Such an alkane may be obtained, directly or in a plurality of steps, from a plant raw material such as an oil, butter or wax, etc. By way of example, suitable alkanes for the invention include the alkanes described in the patent applications of COGNIS WO 2007/068371, or WO2008/155059 (mixtures of separate alkanes differing by at least one carbon). These alkanes are obtained from fatty alcohols, themselves obtained from coconut or palm oil.

It is possible to use the volatile linear alkane alone or preferably to use a mixture of at least two separate volatile linear alkanes, differing from each other by a carbon number n of at least 1, in particular differing from each other by a carbon number of 1 or 2.

By way of example, mixtures suitable for the invention include, in particular, the following mixtures:
50 to 90 wt %, preferably 55 to 80 wt %, more preferably 60 to 75 wt % volatile linear Cn alkane with n ranging from 9 to 13;
10 to 50 wt %, preferably 20 to 45 wt %, preferably 24 to 40 wt %, volatile linear Cn+x alkane with x greater than or equal to 1, preferably x=1 or x=2, with n+x between 10 and 13, relative to the total weight of alkanes in said mixture.

More particularly, a volatile linear alkane suitable for the invention may be used in the form of an n-undecane/n-tridecane (C₁₁/C₁₃) mixture.

In particular, a volatile linear alkane mixture is used, comprising:
55 to 80 wt %, preferably 60 to 75 wt % volatile linear C₁₁ alkane (n-undecane);
20 to 45 wt %, preferably 24 to 40 wt % volatile linear C₁₃ alkane (n-tridecane) relative to the total weight of alkanes in said mixture.

According to yet another embodiment, an n-nonane and n-dodecane (C₉/C₁₂) mixture is used.

The preferred linear alkanes include the following tradenames:
VEGELIGHT SILK from BIOSYNTHIS with INCI name C₉₋₁₂ ALKANE;
VEGELIGHT 1214 LC D from BIOSYNTHIS with INCI name C₉₋₁₂ ALKANE (and) COCO-CAPRYLATE/CAPRATE
CETIOL ULTIMATE MB from BASF with INCI name UNDECANE, TRIDECANE, TOCOPHEROL, HELIANTHUS ANNUUS (SUNFLOWER) SEED OIL.

### Branched Alkanes

These include, in particular isododecane, isodecane, and isohexadecane. Preferably isododecane is used.

According to a particular embodiment, the composition of the invention comprises at least isododecane.

According to a particular embodiment, the volatile hydrocarbon oils are chosen from the group consisting of isododecane, volatile linear alkanes comprising 9 to 13 carbon atoms, and the mixtures thereof.

According to another particular embodiment, the composition of the invention comprises at least one or more volatile linear alkanes.

The volatile oils are present in the composition of the invention in a total amount of 10 to 40 wt %, in particular 10 to 20 wt % relative to the total weight of the composition. The total content of volatile oils also includes the volatile oils used as dispersants and comprised in the other ingredients of the composition, such as the hydrophobic film-forming polymer.

### Non-Volatile Oils

According to an embodiment, the composition according to the invention can further comprise non-volatile oils, in order to improve comfort during application on the keratinous materials.

The non-volatile hydrocarbon oils can include, in particular, hydrocarbon oils, hydrocarbon oils of plant origin, C₁₀-C₄₀ synthesis ethers, C₁₀-C₄₀ synthesis esters, C₁₂-C₂₆ fatty alcohols, fatty acids with greater than C₁₂-C₂₂, and the mixtures thereof.

Non-volatile silicone oils include, in particular, phenyl silicone oils, non-phenyl silicone oils, and the mixtures thereof.

However, according to a particular and preferred embodiment, the total amount of non-volatile oils in the composition of the invention is less than or equal to 5 wt % relative to the total weight of the composition.

According to a particular embodiment, the composition comprises an amount of non-volatile oils less than or equal to 2 wt %, or even less than or equal to 1 wt % relative to the total weight of said composition, or even is devoid of non-volatile oils.

The total content of non-volatile oils also includes the non-volatile oils used as dispersants and comprised in the other ingredients of the composition.

According to a particular embodiment, the composition is devoid of non-volatile oils.

### Hydrophobic Film-Forming Polymer

The composition of the invention also comprises a hydrophobic film-forming polymer.

Film-forming polymer shall mean a polymer capable of forming a continuous film on a support. In the text, the word polymer may designate a homopolymer or a copolymer. The term “copolymer” shall mean a polymer comprising at least two different monomers or two different blocks, which may be from the same chemical family but of different structure. Hydrophobic or liposoluble film-forming polymer shall mean a film-forming polymer dissolved in the oily phase of the composition.

The hydrophobic film-forming polymer can be of natural or synthetic origin, and is advantageously chosen from the group consisting of:
trimethylsiloxysilicates, in particular Belsil TMS 803 (WACKER), KF7312 from Shin Etsu
phenylalkylsiloxysilicates, in which the alkyl group preferably comprises 1 to 6 carbon atoms, such as phenylpropyldimethylsiloxysilicate,
silicone acrylate polymers acrylates such as acrylate/dimethicone copolymers, and in particular acrylate/dimethicone copolymers in cyclopentasiloxane (such as, for example, KP-545 from Shin-Etsu), acrylate/dimethicone copolymers in methyl trimethicone (such as, for example, KP-549 and KP-579 from Shin-Etsu), and acrylate/dimethicone copolymers in isododecane (such as, for example, KP-550 from Shin-Etsu); acrylate/polytrimethylsiloxymethacrylate copolymers and, in particular, acrylate/polytrimethylsiloxymethacrylate copolymers in dimethicone (such as, for example, FA-4003 DM from Dow Corning®), acrylate/polytrimethylsiloxymethacrylate copolymers in isododecane (such as, for example, FA-4004 ID from Dow Corning®),
polyalkylsilsesquioxanes comprising 1 to 6 carbon atoms, and preferably polymethylsilsesquioxane (such as, for example, Silform® Flexible Resin from Momentive),
trialkylsiloxysilylcarbamoyl pullulans, in which the alkyl group comprises 1 to 6 carbon atoms, and preferably trimethylsiloxysilylcarbamoyl pullulan (such as, for example, TSPL-30-ID from Shin-Etsu),
copolymers of vinylpyrrolidone (VP) and alkene comprising 2 to 20 carbon atoms, such as VP/eicosene, VP/hexadecene and VP/styrene copolymers,
copolymers of a vinyl ester, and preferably vinyl acetate/allyl stearate, vinyl acetate/vinyl laurate, vinyl acetate/vinyl stearate, vinyl acetate/vinyl octadecene, vinyl acetate/octadecyl vinyl ether, vinyl propionate/allyl laurate, vinyl propionate/vinyl laurate, vinyl stearate/octadecene-1, vinyl acetate/dodecene-1, vinyl stearate/ethyl vinyl ether, vinyl propionate/cetyl vinyl ether, vinyl stearate/allyl acetate, vinyl dimethyl-2,2-octanoate/vinyl laurate, allyl dimethyl-2,2-pentanoate/vinyl laurate, vinyl dimethyl propionate/vinyl stearate, and allyl dimethyl propionate/vinyl stearate copolymers,
hydrogenated or non-hydrogenated polyolefins and preferably polymers or copolymers of alkenes comprising 2 to 20 carbon atoms, such as the polybutenes, polyisobutenes and polydecenes,
alkyl celluloses, and preferably alkyl celluloses bearing an alkyl group comprising 2 to 6 carbon atoms, such as ethyl cellulose and propyl cellulose,
polyvinyl alcohols, and
the mixtures thereof.

The hydrophobic film-forming polymer is preferably a silicone film-forming polymer chosen from the group consisting of:
trimethylsiloxysilicates,
phenylalkylsiloxysilicates, in which the alkyl group preferably comprises 1 to 6 carbon atoms, such as phenylpropyldimethylsiloxysilicate,
silicone acrylate polymers acrylates such as acrylate/dimethicone copolymers, and in particular acrylate/dimethicone copolymers in cyclopentasiloxane (such as, for example, KP-545 from Shin-Etsu), acrylate/dimethicone copolymers in methyl trimethicone (such as, for example, KP-549 and KP-579 from Shin-Etsu), and acrylate/dimethicone copolymers in isododecane (such as, for example, KP-550 from Shin-Etsu); acrylate/polytrimethylsiloxymethacrylate copolymers and, in particular, acrylate/polytrimethylsiloxymethacrylate copolymers in dimethicone (such as, for example, FA-4003 DM from Dow Corning®), acrylate/polytrimethylsiloxymethacrylate copolymers in isododecane (such as, for example, FA-4004 ID from Dow Corning®),
polyalkylsilsesquioxanes comprising 1 to 6 carbon atoms, and preferably polymethylsilsesquioxane (such as, for example, Silform® Flexible Resin from Momentive),
trialkylsiloxysilylcarbamoyl pullulans, in which the alkyl group comprises 1 to 6 carbon atoms, and preferably trimethylsiloxysilylcarbamoyl pullulan (such as, for example, TSPL-30-ID from Shin-Etsu),
   and the mixtures thereof.

More preferably, the liposoluble film-forming polymer b) is chosen among the trimethylsiloxysilicates, silicone acrylate polymers, and the mixtures thereof.

According to a particular and preferred embodiment, the composition of the invention comprises at least one trimethylsiloxysilicate.

The hydrophobic film-forming polymer can be present in an amount ranging from 1 to 20 wt %, preferably 2 to 15 wt %, better 5 to 12 wt % of dry matter relative to the total weight of said composition. The percentage of hydrophobic film-forming polymer is expressed in wt % of dry extract (dry matter or active matter, a.m.) relative to the total weight of the composition.

### Absorbent Fillers

The term “fillers” should be understood as colourless or white, mineral or synthetic particles of any shape, insoluble in the medium of the composition. These fillers serve in particular to modify the rheology or the texture of the composition and/or provide a matt effect. The fillers can be mineral or organic and of any shape: platelets, spherical or oblong.

The fillers according to the invention are fillers having an ability to absorb and/or adsorb an oil or a liquid fatty substance.

Hence, the absorbent fillers according to the invention can be characterised by their absorption of oil, otherwise termed oil uptake, expressed in millimetres of oil per gram of filler (ml/g).

This oil uptake corresponds to the quantity of oil absorbed and/or adsorbed by the filler and can be characterised by the measurement of oil uptake according to standard methods. These include, in particular, the powder method for determining the oil uptake described in standard NF T 30-022 and corresponding to the quantity of oil adsorbed on the available surface of the filler, expressed as volume of oil adsorbed over the mass of the filler.

According to a particular and preferred embodiment, the following method is used for the oil uptake:
the artificial sebum (25% Jojoba oil, 15% squalane and 60% miglyol 829 (CAPRYLIC/CAPRIC/SUCCINIC TRIGLYCERIDE) is poured onto 1 g of powder (filler to be tested);
with a spatula, the powder is gently pasted by lightly crushing the powder, the objective being to arrive at a mastic paste that does not crumble and peels off easily;
The result is recorded in ml of artificial sebum per 1 g of powder. It must be converted into ml/100 g by multiplying the value read by 100;
the measurement is carried out twice on each sample and if the difference between these two measurements is greater than 0.05 ml, a third measurement is carried out.

According to a particular embodiment, the fillers further have a low capacity for absorbing and/or adsorbing water.

The same method is used for water uptake, replacing the artificial sebum by demineralised water.

In practice, it is possible to use:
a 10-ml burette (precision 0.05 ml) filled with demineralised water for the water uptake,
a 10-ml burette (precision 0.05 ml) filled with artificial sebum as described above for the oil uptake,
a large diameter watch glass, on which 1 g of powder (filler to be tested) is weighed,
a balance, and
a wide flat spatula with round end.

The larger the value of the volume poured, the greater the capacity of the filler to absorb water or sebum.

If the filler does not absorb any water at all, then it is hydrophobic.

If the water does not absorb any sebum at all, then it is lipophobic (with respect to artificial sebum).

According to a particular embodiment of the invention, fillers are used which have an oil uptake capacity of at least 30 ml/100 g.

According to a particular embodiment of the invention, fillers are used which have an oil uptake capacity of between 55 ml/100 g and 250 ml/100 g.

According to a particular embodiment of the invention, fillers are used which have an oil uptake capacity of between 30 ml/100 g and 250 ml/100 g, in particular between 55 ml/100 g and 250 ml/100 g.

According to a particular embodiment of the invention, the fillers also have a water uptake capacity less than 300 ml/100 g, in particular between 0 ml/100 g and 250 ml/100 g, in particular between 0 ml/100 g and 170 ml/100 g.

In particular, among these fillers additionally having a water uptake capacity less than 300 ml/100 g, it is possible to use:
a filler having a water uptake capacity between 55 ml/100 g and 100 ml/100 g and a zero water uptake, such as the filler marketed under the name AMIHOPE LL MB;
a filler having an oil uptake capacity between 120 ml/100 g and 145 ml/100 g and a water uptake between 120 ml/100 g and 150 ml/100 g such as the filler marketed under the name AMILON.

Non-limiting examples of fillers that can be used according to the invention are described in the table 1 below:

TABLE 1

| Fillers | | Oil | Water |
|---|---|---|---|
| (commercial | | uptake | uptake |
| reference) | INCI name | (ml/100 g) | (ml/100 g) |
| ORGASOL 2002 | NYLON-12 | 55 | 105 |
| DNAT COS | | | |
| (reference filler) | | | |
| MAKIBEADS 150 | METHYL | 175 | 0 |
| | METHACRYLATE | | |
| | CROSSPOLYMER | | |
| | (PMMA) | | |
| BORON NITRIDE | BORON NITRIDE | 95 | 105 |
| CC6058 | | | |
| AMILON | SILICA AND | 130 | 145 |
| | AMINO ACID | | |
| AMIHOPE LL MB | LAUROYL LYSINE | 75 | 0 |
| RAMIE CELLULOSE | CELLULOSE | 75 | 150 |
| POWDER | | | |
| ORGASOL POUDRE | NYLON-12 in | 60 | 0 |
| EXP HCO | HYDROGENATED | | |
| | CASTOR OIL | | |
| MK-7 | SILICA | 245 | 260 |
| SPHERICA BA-1 | SILICA | 150 | 265 |
| RONAFLAIR | SILICA AND | 50 | 60 |
| FLAWLESS | TITANIUM DIOXIDE | | |
| | (CI 77891) | | |
| | AND IRON OXIDES | | |
| | (CI 77491) | | |
| RAMIE CELLULOSE | CELLULOSE | 75 | 150 |
| POWDER | | | |
| SILK COTTON PW | CELLULOSE | 120 | 260 |

Hence the filler or fillers according to the invention are chosen, in particular, among:
polyamides powders (e.g. nylon)
acrylic polymer powders, in particular poly(methyl methacrylate) (PMMA)
boron nitride powders
silica and amino acid powders
lauroyl lysine,
silica powders, microspheres of amorphous silica, silica microbeads,
cellulose powders, cellulose beads,
starch powders,
cross-linked organopolysiloxane elastomer powders, coated or not coated with silicone resin,
silicone resin powders (INCI name polymethylsilsesquioxane)
silicone resin/TiO2 powders,
polyurethane and silica powders,
calcium carbonate,
clays
   and the mixtures thereof.

According to a particular embodiment, the filler or fillers according to the invention are chosen from the group consisting of polyamide powders (e.g. nylon), acrylic polymer powders, in particular poly(methyl methacrylate) (PMMA) powders, boron nitride powders, silicon and amino acid powders, lauroyl lysine, silica powders, cellulose powders, cross-linked organopolysiloxane elastomer powders, silicone resin powders, starch powders and the mixtures thereof, preferably silica and amino acid powders, lauroyl lysine, and the mixture thereof.

The total amount of absorbent fillers according to the invention will be, in particular, 1 to 10 wt %, in particular 2 to 8 wt % and preferably 3 to 5 wt % relative to the total weight of said composition.

### Pigments

The composition of the invention further comprises at least one pigment.

“Pigments” shall mean mineral or organic, white or coloured particles, insoluble in an aqueous solution, intended for colouring and/or opacifying the resulting deposit. These can include mineral pigments, organic pigments and composite pigments (in other words pigments based on mineral and/or organic materials).

According to a particular embodiment, the pigment or pigments are chosen, in particular, among mineral and/or organic pigments, composite pigments (based on mineral and/or organic materials), mother-of-pearl or pearlescent pigments, and the mixtures thereof.

The “mineral pigments” can include, by way of example, titanium dioxide (rutile or anatase), optionally surface treated; black, yellow, red and brown iron oxides; manganese violet; ultramarine blue, chromium oxide, hydrated chromium oxide and ferric blue.

The “organic pigments” can include, for example, pigments D & C red no. 19; D & C red no. 9; D & C Red no. 22; D & C Red no. 21; D & C Red no. 28; D & C Yellow no. 6; D & C orange no. 4; D & C orange no. 5; D & C Red no. 27; D & C red no. 13; D & C Red no. 7; D & C Red no. 6; D & C Yellow no. 5; D & C Red no. 36; D & C Red no. 33; D & C orange no. 10; D & C yellow no. 6; D & C Red no. 30; D &C red no. 3; D &C Blue 1; carbon black and cochineal carmine based lacquers.

The pigments are advantageously surface treated by at least one hydrophobic or lipophilic treatment agent for improved dispersion in the oily phase. The hydrophobic treatment agent is chosen, in particular, from the group consisting of silicone surface agents; fluorinated surface agents; fluorosilicone surface agents; metal soaps, N-acyl amino acids or their salts; lecithin and the derivatives thereof; isopropyl trisostearyl titanate; diisostearyl sebacate; natural plant or animal waxes, synthetic polar waxes; fatty esters; phospholipids, and the mixtures thereof.

In particular, the one or more pigments are present in the composition in an amount ranging from 4% to 30 wt %, preferably 8% to 20 wt % relative to the total weight of the composition.

According to a particular embodiment, the cosmetic make-up composition for the skin in the form of a water-in-oil emulsion (W/O) comprises, in a physiologically acceptable medium, at least:

a) an aqueous dispersion of polyurethane, preferably with INCI name ‘Polyurethane-35’ or ‘Polyurethane-35 and water’, in an amount ranging from 0.2 to 20 wt %, in particular 0.5 to 15%, preferably 1 to 10%, more preferably 1 to 7 wt % of dry matter (polyurethane) relative to the total weight of said composition,
b) a hydrophobic film-forming polymer, preferably chosen among the trimethylsiloxysilicates, silicone acrylate polymers and the mixtures thereof, in an amount ranging from 1 to 20 wt %, preferably 2 to 15 wt %, more preferably 5 to 12 wt % of dry matter relative to the total weight of said composition,
c) one or more volatile hydrocarbon oils, preferably in an amount of at least 10 wt % relative to the total weight of the composition,
d) one or more absorbent fillers having an oil uptake greater than or equal to 30 ml/100 g, chosen in the group consisting of the polyamides powders (e.g. nylon), acrylic polymer powders, in particular poly(methyl methacrylate) (PMMA), boron nitride powders, silica and amino acid powders, lauroyl lysine, silica powders, cellulose powders, cross-linked organopolysiloxane elastomer powders, silicone resin powders, starch powders, preferably silica and amino acid powders, lauroyl lysine, and the mixtures thereof in a total amount ranging from 1 to 10 wt %, in particular 2 to 8 wt % and preferably 3 to 5 wt % relative to the total weight of said composition,
e) pigments.

### Galenic

The composition is preferably intended to be applied on the skin, in particular on the skin of the face and/or neck and is preferably in the form of a water-in-oil emulsion (W/O).

The composition is, for example, in the form of a fluid for the face, a foundation, a foundation base or a ‘finisher’. It is preferably a composition for make-up of the face, in particular a foundation.

The composition of the invention may also include any additive usually used in cosmetics, such as UV filters, antioxidants, surfactants, gelling agents, preservatives, film-forming polymers, perfumes, cosmetic active ingredients, such as, for example, emollients, moisturisers, vitamins, anti-ageing agents, lightening agents, and the mixtures thereof.

### Cosmetic Method

The invention also relates to a cosmetic method for make-up of keratinous materials, in particular the skin, preferably the skin of the face and/or the neck, comprising applying, on said keratinous material, at least one cosmetic composition such as previously defined in the invention.

In particular, the method is intended to confer an improved wear of the make-up over time, in particular in extreme conditions, in particular when the composition of the invention is applied on an oily skin and/or subject to hot and/or humid atmospheric conditions, and/or sweating conditions linked to intense physical exercise (e.g. practising a sport).

The invention will be illustrated in the following non-limiting examples. Unless indicated otherwise, the % are expressed in wt % relative to the total weight of said composition.

### EXAMPLES

The inventors have worked to optimise the wear properties, in particular wear of the mattness, of a foundation emulsion as described above, for better resistance to extreme conditions (e.g. heat/humidity or practising sport).

TABLE 2

| Ingredients | % |
|---|---|
| Isododecane | 14% |
| Silicone resin (lipophilic | 9% AM |
| polymer according to the invention) | |
| Surfactants | 3% |
| Titanium dioxide and | 12% |
| iron oxides (pigments) | |
| Volatile oil according | 6% |
| to the invention | |
| Absorbent filler according | 4% |
| to the invention | |
| Aqueous dispersion of | 4% AM |
| polyurethane (hydrophilic polymer | |
| according to the invention) | |
| Glycols | 6% |
| Ethanol | 8% |
| Preservatives | 0.8% |
| Water, quantity sufficient for | 100% |

### Example 1: Selection of Volatile Oils Suitable for the Invention

### Test Protocol:

20 mg of the oil to be studied is weighed on a plate of PMMA of 5 cm×5 cm using a micropipette and a precision balance. This material is spread by finger over the entire plate. Said plate is then deposited in a thermostatic ventilated chamber at 25° C. and 50% humidity. For this material, the test is performed 3 times in order to calculate a standard deviation and to have an idea of the repeatability of the method.

The mass loss during drying is measured after 15 minutes, 30 minutes and 60 minutes. The mass loss is expressed according to the following calculation:

$$\% mass loss = \frac{{m}_{t 0} - {m}_{tx}}{{m}_{t 0}} \times 100 \qquad [ Math . 2 ]$$

with mtx corresponding to the mass remaining at the measurement time (t15 min, t30 min or t60 min) and mt0 corresponding to the initially applied mass.
Four volatile oils have been tested and compared to a reference oil:

TABLE 3

| Oils | INCI Name |
|---|---|
| Oil 1 (Reference oil) | CYCLOPENTASILOXANE (70%)/ |
| | CYCLOHEXASILOXANE (30%) |
| Oil 2 | ISODODECANE |
| Oil 3 | UNDECANE (65%)/TRIDECANE (35%) |
| Oil 4 | VOLATILE PENTACYCLOMETHICONE |
| Oil 5 | ISOHEXADECANE |

The mass loss measurement results, expressed as a percentage, are presented in table 4 below:

TABLE 4

| | | Evaporation time (minutes) | | | |
|---|---|---|---|---|---|
| | | 0 | 15 | 30 | 60 |
| Oil 1 (reference) | Mean | 0.0% | 41.8% | 69.2% | 90.0% |
| | Standard | 0.0% | 5.1% | 4.7% | 5.6% |
| | deviation | | | | |
| Oil 2 | Mean | 0.0% | 100.0% | 100.0% | 100.0% |
| | Standard | 0.0% | 1.1% | 0.2% | 0.8% |
| | deviation | | | | |
| Oil 3 | Mean | 0.0% | 75.7% | 87.9% | 99.2% |
| | Standard | 0.0% | 4.7% | 4.8% | 4.4% |
| | deviation | | | | |
| Oil 4 | Mean | 0.0% | 64.8% | 99.7% | 100.0% |
| | Standard | 0.0% | 5.3% | 1.6% | 1.5% |
| | deviation | | | | |
| Oil 5 | Mean | 0.0% | 22.3% | 42.2% | 76.2% |
| | Standard | 0.0% | 5.1% | 7.5% | 11.7% |
| | deviation | | | | |

The results show significant differences in volatility between the five oils tested. ISODODECANE is the most volatile of all the oils, being totally evaporated after only 15 minutes of drying.

With regard to the linear alkane UNDECANE (65%)/TRIDECANE (35%) and VOLATILE PENTACYCLOMETHICONE, the evaporation curves are rather close.

CYCLOPENTASILOXANE (70%)/CYCLOHEXASILOXANE (30%) (reference oil 1) is less volatile than oils 2, 3 and 4. Even after 60 minutes, a little oil still remains on the PMMA plate. ISOHEXADECANE is less volatile than all the other oils and is considered according to the invention to be the lower limit of volatility.

In the context of the invention, volatile oil means an oil having lost more than 20 wt % of its mass after 15 minutes, more than 40 wt % of its mass after 30 minutes and more than 70 wt % of its mass after 60 minutes, according to the protocol described above.

The compositions of the invention advantageously comprise volatile hydrocarbon oils chosen among the volatile linear C9-C15 alkanes, isododecane, and the mixtures thereof.

The use of undecane/tridecane is particularly advantageous in a composition of the invention as described in table 2. Compared to similar compositions comprising volatile silicone oils, it improves the adherence and mattness of the film of foundation applied on the skin.

### Example 2: Selection of Fillers Suitable for the Invention

The study aims to test fillers in a wide range of sebum uptake with a strong preferential affinity for oil, sebum being suspected of being one of the most impacting factors on the stability performance of the foundation film.

### Test Protocol:

the artificial sebum (25% Jojoba oil, 15% squalane and 60% miglyol 829 (CAPRYLIC/CAPRIC/SUCCINIC TRIGLYCERIDE) is poured onto 1 g of powder (filler to be tested);
with a spatula, the powder is gently pasted by lightly crushing the powder, the objective being to arrive at a mastic paste that does not crumble and peels off easily;
The result is recorded in ml of artificial sebum per 1 g of powder. It must be converted into ml/100 g by multiplying the value read by 100;
the measurement is carried out twice on each sample and if the difference between these two measurements is greater than 0.05 ml, a third measurement is carried out.

The same method is used for water uptake, replacing the artificial sebum by demineralised water.

In practice, it is possible to use:
a 10-ml burette (precision 0.05 ml) filled with demineralised water for the water uptake,
a 10-ml burette (precision 0.05 ml) is filled with artificial sebum as described above for the oil uptake,
a large diameter watch glass, on which 1 g of powder (filler to be tested) is weighed,
a balance, and
a wide flat spatula with round end.

The larger the value of the volume poured, the greater the capacity of the filler to absorb water or sebum.

If the filler does not absorb any water at all, then it is hydrophobic.

If the water does not absorb any sebum at all, then it is lipophobic (with respect to artificial sebum).

The fillers tested and the values obtained are presented in table 5 below:

TABLE 5

| Fillers | | Oil | Water |
|---|---|---|---|
| (commercial | | uptake | uptake |
| reference) | INCI name | (ml/100 g) | (ml/100 g) |
| ORGASOL 2002 | NYLON-12 | 55 | 105 |
| DNAT COS | | | |
| MAKIBEADS 150 | METHYL | 175 | 0 |
| | METHACRYLATE | | |
| | CROSSPOLYMER | | |
| | (PMMA) | | |
| BORON NITRIDE | BORON NITRIDE | 95 | 105 |
| CC6058 | | | |
| AMILON | SILICA AND | 130 | 145 |
| | AMINO ACID | | |
| AMIHOPE LL MB | LAUROYL LYSINE | 75 | 0 |
| RAMIE CELLULOSE | CELLULOSE | 75 | 150 |
| POWDER | | | |
| ORGASOL POUDRE | NYLON-12 in | 60 | 0 |
| EXP HCO | HYDROGENATED | | |
| | CASTOR OIL | | |
| MK-7 | SILICA | 245 | 260 |
| SPHERICA BA-1 | SILICA | 150 | 265 |
| RONAFLAIR | SILICA AND | 50 | 60 |
| FLAWLESS | TITANIUM DIOXIDE | | |
| | (CI 77891) | | |
| | AND IRON OXIDES | | |
| | (CI 77491) | | |
| RAMIE CELLULOSE | CELLULOSE | 75 | 150 |
| POWDER | | | |
| SILK COTTON PW | CELLULOSE | 120 | 260 |
| CHIFFONSIL 5T | SILICA AND | 15 | 20 |
| (outside of | TITANIUM | | |
| the invention) | DIOXIDE | | |
| COVABEAD CRYSTAL | CALCIUM SODIUM | 20 | 20 |
| (outside of | BOROSILICATE | | |
| the invention) | | | |

Fillers having an oil uptake less than 30 ml/100 g are not kept because they do not absorb sufficient sebum.

The absorbent fillers according to the invention should therefore have an oil uptake of at least 30 ml/100 g.

According to a particular embodiment of the invention, fillers are used which have an oil uptake capacity of at least 30 ml/100 g.

According to a particular embodiment, the filers can additionally have a water uptake capacity less than 300 ml/100 g.

According to a particular embodiment of the invention, fillers are used which have an oil uptake capacity of between 55 ml/100 g and 250 ml/100 g, and a water uptake capacity between 0 ml/100 g and 170 ml/100 g.

### Example 2: Formulations and Wear Performance

The compositions illustrated below are prepared according to the conventional methods for formulations in the cosmetic field. The % are expressed in wt % of raw material, unless indicated otherwise.

### 2-1 Long Lasting Water-In-Oil Foundation

TABLE 6

| Ingredients | % |
|---|---|
| Isododecane (volatile oil) | 12.4% |
| Trimethylsiloxysilicate | 9.6% AM |
| BELSIL TMS 803 (lipophilic | |
| film-forming polymer) | |
| PEG-10 dimethicone | 3.0% |
| Titanium dioxide and iron oxides | 12% |
| Undecane/tridecane | 6.1% |
| (CETIOL Ultimate) | |
| (volatile oil) | |
| SILICA AND AMINO | 4.0% |
| ACID (AMILON ) | |
| Glycols | 6.0% |
| Aqueous dispersion of | 4.0% AM |
| polyurethane (BAYCUSAN 1004] | |
| (hydrophilic film-forming polymer) | |
| Alcohol | 8.0% |
| Preservatives | 0.8% |
| Water quantity sufficient for | 100% |

### 2-2 Long Lasting Water-In-Oil Foundation

TABLE 7

| Ingredients | % |
|---|---|
| Isohexadecane | 3% |
| Acrylate/polytrimethylsiloxy- | 9.6% AM |
| methacrylate copolymer | |
| (DC 4004 ID) (lipophilic | |
| film-forming polymer) | |
| PEG-10 dimethicone | 3.0% |
| Titanium dioxide | 12% |
| and iron oxides | |
| Dodecane/tetradecane | 15% |
| (VEGELIGHT) | |
| METHYL METACRYLATE | 4.0% |
| CROSSPOLYMER (MAKIBEADS 150) | |
| Glycols | 6.0% |
| Aqueous dispersion of | 4.0% AM |
| polyurethane (BAYCUSAN 1004] | |
| (hydrophilic film-forming polymer) | |
| Alcohol (ethanol) | 8.0% |
| Preservatives | 0.8% |
| Water quantity sufficient for | 100% |

### 2-3 Long Lasting Water-In-Oil Foundation

TABLE 8

| Ingredients | % |
|---|---|
| Undecane/tridecane | 7% |
| (CETIOL Ultimate) | |
| (volatile oil) | |
| Polymethylsilsesquioxane | 9.6% AM |
| (Silform Flexible resin) | |
| (lipophilic film-forming | |
| polymer) | |
| PEG-10 dimethicone | 3.0% |
| Titanium dioxide and iron oxides | 12% |
| Undecane/tridecane | 6.1% |
| (CETIOL Ultimate) | |
| (volatile oil) | |
| SILICA AND AMINO | 2.0% |
| ACID (AMILON) | |
| LAUROYL LYSINE | 2.0% |
| (AMIHOPE LB) | |
| Glycols | 6.0% |
| Aqueous dispersion of | 4.0% AM |
| polyurethane (BAYCUSAN 1004] | |
| (hydrophilic film-forming polymer) | |
| Alcohol (ethanol) | 8.0% |
| Preservatives | 0.8% |
| Water quantity sufficient for | 100% |

### Wear Performance of the Mattness

Composition 2-2 of the invention has been evaluated according to various criteria:
presence of the foundation film (Visia photograph under UV light and calculation of the parameter L* using image analysis),
homogeneity of the foundation film (Visia photograph under crossed polarised light and calculation of the parameter σ76 using image analysis),
mattness of the foundation film (gloss measurement at 60° with the Glossmeter),
foundation transfer properties (Texturometer and analyse of the image for the colorimetric difference compared to the white reference (cotton white),
foundation coverage (colour measurement using a spectrocolorimeter).

The quantity of foundation applied is calibrated.

The foundation according to the invention makes it possible to obtain good wear properties, in particular wear of the mattness, comfort after application and non-transfer.

### Wear Performance Under Extreme Conditions

Composition 2-2 has also been evaluated under extreme conditions, simulating hot and humid atmospheric conditions and sweating conditions linked to practising a sport. Two different tests have been carried out based on a panel of 22 women.

The first test involved submitting the foundation to a plurality of 10-minute, heat and humidity cycles using a halogen lamp and monitoring the wear over an eight-hour day. For this purpose, the naked-skin face of the panelist was evaluated in a plurality of ways: through clinical evaluation by a dermatologist, through taking a photograph and through self-evaluation by the panelist. Once the naked skin evaluation is carried out, the foundation is applied on the entire face of the panelist. The face of the panelist is then evaluated again (at t intermediate) by means of a photograph, the dermatologist and self-evaluation. Then, the panelist is subjected to four heat and humidity cycles applied at different times: 1 hour, 3 hours, 5 hours and 7 hours after application of the foundation. The change in the foundation is monitored at times t4 h, t6 h and t8 h.

The second test consists of evaluating the resistance of the foundation under sport conditions. For this purpose, after application of the foundation, the women practice a self-evaluation and respond to a questionnaire treating different parameters of the applied foundation film. The women then cycle for a period of 30 to 45 minutes and re-evaluate the foundation by performing a new self-evaluation and responding to a second questionnaire.

Results Associated with the Hot and Humid Cycles

The instrumental measurements show a very good wear of the surface of the product and of the colour intensity up to 8 hours. The clinical evaluation by a dermatologist shows an improvement in the homogeneity of the complexion and the texture of the skin, as well as an increase in the radiance of the skin and the brightness of the complexion for up to 8 hours. Finally, the skin is more matt for up to 8 hours. The panelists, like the dermatologist, estimate that the homogeneity and brightness of the complexion are improved for up to 8 hours, and similarly for the perception of skin that is more matt for up to 8 hours.

Results Associated with the Sport Conditions

Just after application of the foundation, up to 91% of the panelists estimate that their skin is matt.

After the sporting activity (45 minutes cycling), the panelists estimate that:
“The product is long-lasting” (95% of the women);
“My complexion remains perfectly the same after my sporting activity” (77% of the women);
“The imperfections and irregularities of the complexion remain corrected even after my activity” (82% of the women);
“The colour has good wear; it does not change after my sporting activity” (86% of the women);
“My complexion remains unified, even after my sporting activity” (77% of the women).

These results, obtained under extreme conditions, therefore show that the compositions according to the invention, in the form of a lipophilic film-forming polymer, an aqueous dispersion of polyurethane, and absorbent fillers having an oil uptake greater than or equal to 30 ml/100 g, in particular greater than or equal to 55 ml/100 g, provides good wear over time, including under extreme conditions (hot and/or humid atmospheric conditions or intense practice of sport).

1. 12. (canceled)

13. A cosmetic make-up composition for the skin in the form of a water-in-oil (W/O) emulsion comprising, in a physiologically acceptable medium, at least:
a) an aqueous polyurethane dispersion,
b) a hydrophobic film-forming polymer,
c) one or more volatile hydrocarbon oils,
d) one or more absorbent fillers having an oil uptake greater than or equal to 30 ml/100 g and
e) pigments.

14. The cosmetic composition according to claim 13, wherein the volatile hydrocarbon oil(s) is (are) in an amount of at least 10 wt % relative to the total weight of the composition.

15. The cosmetic composition according to claim 13, wherein it comprises an amount of non-volatile oils less than or equal to 5 wt %% relative to the total weight of said composition.

16. The cosmetic composition according to claim 13, wherein the absorbent fillers are chosen from the group consisting of polyamide powders (e.g. nylon), acrylic polymer powders, boron nitride powders, silicon and amino acid powders, lauroyl lysine, silica powders, cellulose powders, cross-linked organopolysiloxane elastomer powders, silicone resin powders, starch powders and the mixtures thereof.

17. The cosmetic composition according to claim 16, wherein the absorbent fillers are chosen from the group consisting of silica and amino acid powders, lauroyl lysine, and the mixture thereof.

18. The cosmetic composition according to claim 16, wherein the total amount of absorbent filler(s) is 1 to 10 wt % relative to the total weight of said composition.

19. The cosmetic composition according to claim 13, wherein the dispersed polyurethane comprises the reaction products of:
A) a prepolymer according to formula 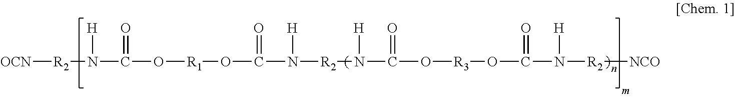
wherein
R1 represents a bivalent radical of a dihydroxyl compound,
R2 represents a hydrocarbon radical derived from an aliphatic or cycloaliphatic polyisocyanate,
R3 represents a hydrocarbon radical derived from a low molecular weight diol,
n has a value of 0 to 5, and
m is >1;
B) at least one chain extender according to the formula:
H₂N—R₄—NH₂ [Chem. 2]
wherein R4 represents an alkylene or alkylene oxide radical that is not substituted by ionic or potentially ionic groups; and
C) at least one chain extender according to the formula:
H₂N—R₅—NH₂ [Chem. 3]
wherein R5 represents an alkylene radical substituted by ionic or potentially ionic groups.

20. The cosmetic composition according to claim 19, wherein:
the radical R1 is obtained by polycondensation of at least one dicarboxylic acid with at least one diol;
the radical R2 is derived from an aliphatic or cycloaliphatic polyisocyanate;
the radical R3 is derived from neopentyl glycol;
the chain extender according to formula Chem. 2 is chosen among ethylene diamine, diethanolamine and the mixtures thereof; and
the chain extender according to formula Chem. 3 is chosen among the diaminosulfonates.

21. The cosmetic composition according to claim 20, wherein:
the radical R1 is obtained by polycondensation of an adipic acid with a diol chosen among hexanediol, neopentyl glycol, and the mixtures thereof;
the radical R2 is derived from a diisocyanate chosen among 1,6-hexamethylene diisocyanate, isophorone diisocyanate and dicyclohexylmethane diisocyanate, and the mixtures thereof, and
the chain extender according to formula Chem. 3 is the sodium salt of N-(2-aminoethyl)-2-aminoethanesulfonic acid.

22. The cosmetic composition according to claim 19, wherein the dispersed polyurethane is present in an amount ranging from 0.2 to 20 wt %, of dry matter relative to the total weight of said composition.

23. The cosmetic composition according to claim 13, wherein the hydrophobic film-forming polymer is a hydrophobic film-forming silicone polymer chosen from the group consisting of trimethylsiloxysilicates, phenylalkylsiloxysilicates in which the alkyl group preferably comprises 1 to 6 carbon atoms, silicone acrylate polymers, polyalkylsilsesquioxanes comprising 1 to 6 carbon atoms, and the mixtures thereof.

24. The cosmetic composition according to claim 23, wherein the hydrophobic film-forming silicone polymer is chosen from trimethylsiloxysilicates.

25. The composition according to claim 23, wherein that hydrophobic film-forming polymer is present in an amount ranging from 1 to 20 wt %, of dry matter relative to the total weight of said composition.

26. The cosmetic composition according to claim 13, wherein the volatile hydrocarbon oils are chosen from the group consisting of isododecane, volatile linear alkanes comprising 9 to 13 carbon atoms, and the mixtures thereof.

27. A cosmetic method for make-up of the skin comprising the application on the skin of a composition as defined in claim 13.

28. The cosmetic method according to claim 27, wherein the composition is applied to oily skin and/or skin subject to hot and/or humid atmospheric conditions, and/or sweating conditions associated with intense physical exercise.
